(19) United States
(12) Patent Application Publication
(10) Pub. No.: US 2006/0217657 A1
(43) Pub. Date: Sep. 28, 2006

(54) Surgical suction device

(76) Inventor: Richard Yacowitz, Little Silver, NJ (US)

Correspondence Address: WALTER J, TENCZA JR., 10 STATION PLACE, SUITE 3, METUCHEN, NJ, 08840, US

(21) Appl. No.: 11/088,869

(22) Filed: Mar. 24, 2005

(51) Int. Cl. A61M 1/00

(52) U.S. Cl. 604/35

(57) ABSTRACT

An apparatus including a perforated top and a spoon is provided. The perforated top may include a bowl portion having a plurality of perforations and a stem connected to the bowl portion. The spoon may include a tube portion and a bowl device connected to the tube portion. The bowl portion of the perforated top is adapted to be placed over the bowl device of the spoon and connected to the bowl device in a manner, which allows liquid to pass through the plurality of perforations, into a portion of the bowl device, and thereafter into the tube portion of the spoon.

### FIELD OF THE INVENTION

This invention relates to improved methods and apparatus concerning suction tools for surgery.

### BACKGROUND OF THE INVENTION

Hollow organs such as the bladder and the gallbladder, and large cysts require the use of straight or angled suction devices to remove fluid during surgical procedures. After the fluid is removed, the contents of these organs or structures, such as stones, sediment, fibrin, clots, degradation, or debris are typically manually removed. The suction devices may become clogged, such as by fat, requiring repeated cleaning of the suction devices. This frequently occurs during surgical procedures involving suctioning of the abdominal cavity omentum.

### SUMMARY OF THE INVENTION

The present invention, in one or more embodiments provides an apparatus including a perforated top and a spoon. The perforated top may include a bowl portion having a plurality of perforations and a stem connected to the bowl portion. The spoon may include a tube portion and a bowl device connected to the tube portion. The bowl portion of the perforated top is adapted to be placed over the bowl device of the spoon and connected to the bowl device in a manner, which allows liquid to pass through the plurality of perforations, into a portion of the bowl device, and thereafter into the tube portion of the spoon.

The spoon may include a slot and the stem of the perforated top may be adapted to fit within the slot when the bowl portion of the perforated top is placed over the bowl device of the spoon and when the bowl portion of the perforated top is connected to the bowl device. The spoon may also include a sleeve, which is adapted to slide over a portion of the stem of the perforated top in order to hold the perforated top onto the spoon.

The spoon may include a connection device connected to the tube portion. The connection device may be adapted to connect the spoon to a suction tube. The suction tube may be connected to a suction or vacuum device typically having a motor and providing suction.

The present invention also includes a method comprising providing a perforated top including a bowl portion having a plurality of perforations, and providing a spoon including a tube portion and a bowl device connected to the tube portion.

### BRIEF DESCRIPTION OF THE DRAWINGS

FIG. 1 shows a perspective view of an apparatus comprised of a spoon and a perforated top, in accordance with an embodiment of the present invention, with the apparatus shown in a disassembled state;

FIG. 2 shows a perspective view of the apparatus of FIG. 1 in an assembled state;

FIG. 3 shows a perspective view of the apparatus of FIG. 1 in an assembled state and a sleeve moved into a position in which it holds the perforated top onto the spoon;

FIG. 4A shows a top view of the perforated top;

FIG. 4B shows a side view of the perforated top;

FIG. 5 shows a perspective view of a a bowl device of the spoon of FIG. 1;

FIG. 6 shows a perspective view of the apparatus of FIG. 3, attached to a tube, which is attached to a suction device.

### DETAILED DESCRIPTION OF THE DRAWINGS

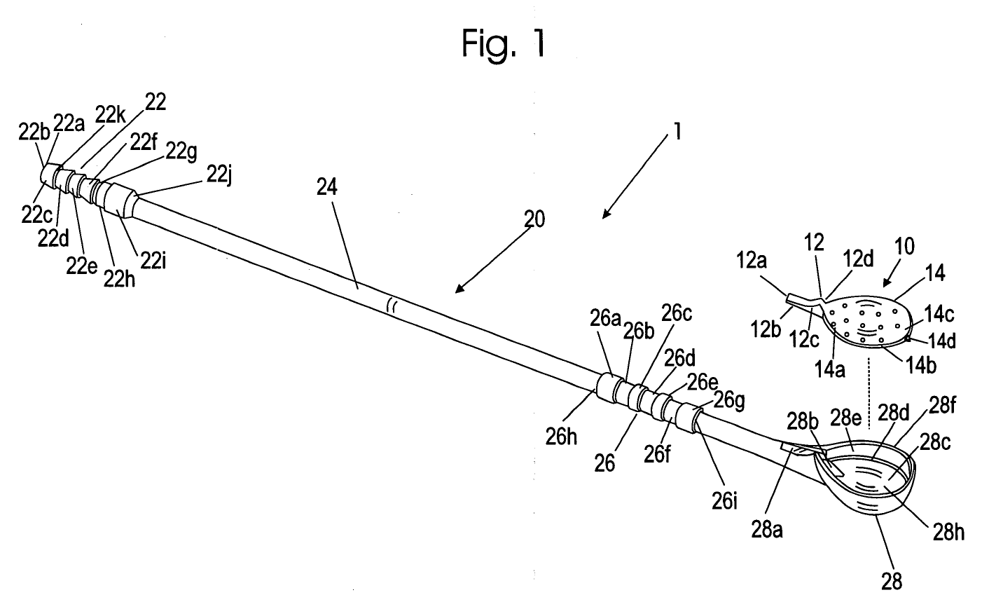

FIG. 1 shows a perspective view of an apparatus 1 comprised of a spoon 20 and a perforated top 10, in accordance with an embodiment of the present invention, with the apparatus 1 shown in a disassembled state.

The spoon 20 includes a connection device 22, a tube 24, a sleeve 26, and a bowl device 28. The connection device 22 may be a hollow tube having a uniform inner diameter. The connection device 22 includes an end 22a, at which is located an opening 22b leading into the hollow inside of the connection device 22. Located on the outside of the connection device 22 are outer smooth barbs or portions 22c, 22d, 22e, and 22f. The barb or portion 22c, increases in outer diameter from the end 22a to the end 22k. The barbs or portions 22d, 22e, and 22f, may be identical to the barb or portion 22c.

The connection device 22 includes portion 22g having an outer diameter, which increases in a direction towards the bowl device 28, and a portion 22h, whose outer diameter is substantially uniform. The connection device 22 also includes portion 22i, which is uniform in outer diameter, and portion 22j, which decreases in outer diameter in the direction towards the bowl device 28.

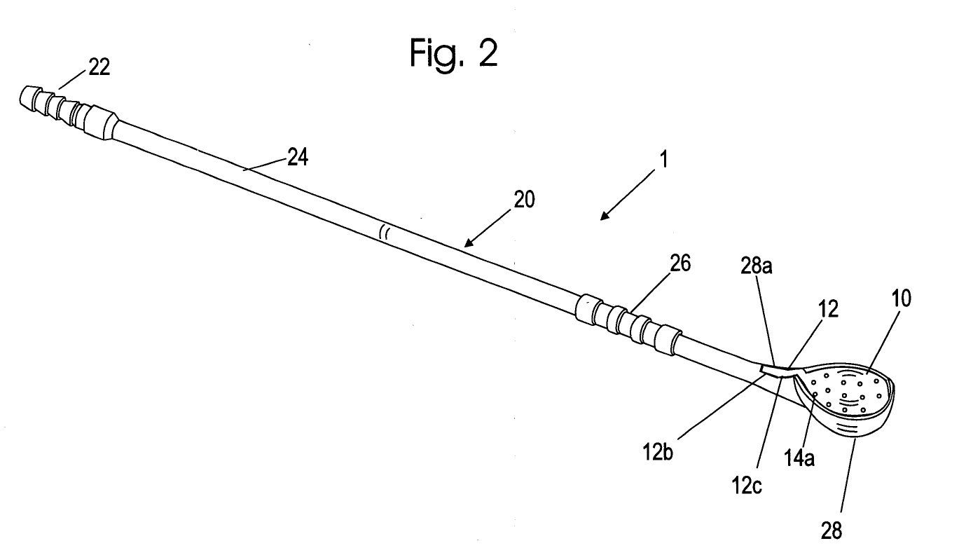

The tube 24 is a hollow tube. The sleeve 26 is a hollow tube, which can slide up and down the tube 24, with an appropriate portion of the tube 24 residing inside the sleeve 26. The sleeve 26 has portions 26a, 26b, 26c, 26d, 26e, 26f, and 26g. Portions 26a-g provide contoured ribbing which allow a surgeon to better grasp the sleeve 26 in order to push the sleeve 26 to release or secure the perforated top 10 to the bowl device 28 of the spoon 20. The sleeve 26 has ends 26h and 26i. The bowl device 28 includes a slot 28a, an opening 28b, a bowl portion 28c, a inner ridge 28d, an inner peripheral surface 28e, and a rim 28f. The bowl device 28 includes a indentation 28g in the surface 28e. The bowl device 28 has an elliptical opening 28h at its top, which is slightly smaller than an elliptical periphery, outline, or edge of the bowl portion 14. When the bowl portion 14 is placed over the elliptical opening 28h, as in FIG. 2, the bowl portion 14 covers the elliptical opening 28h in a snug fit.

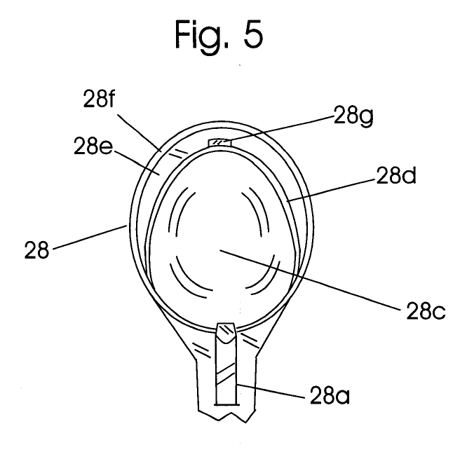

The perforated top 10 includes a handle or stem 12. The stem 12 has an end 12a, a substantially thin flat portion 12b, an inclined portion 12c, and a declining portion 12d. The stem 12 is connected to a bowl portion 14. The bowl portion 14 includes a plurality of perforations, such as perforation 14a. The bowl portion 14 has an edge 14b, and a concave surface 14c. The bowl portion 14 also has a protrusion 14d which can be inserted into a indentation, recess, or slot 28g, shown in FIG. 5, in the surface 28e of the bowl device 28.

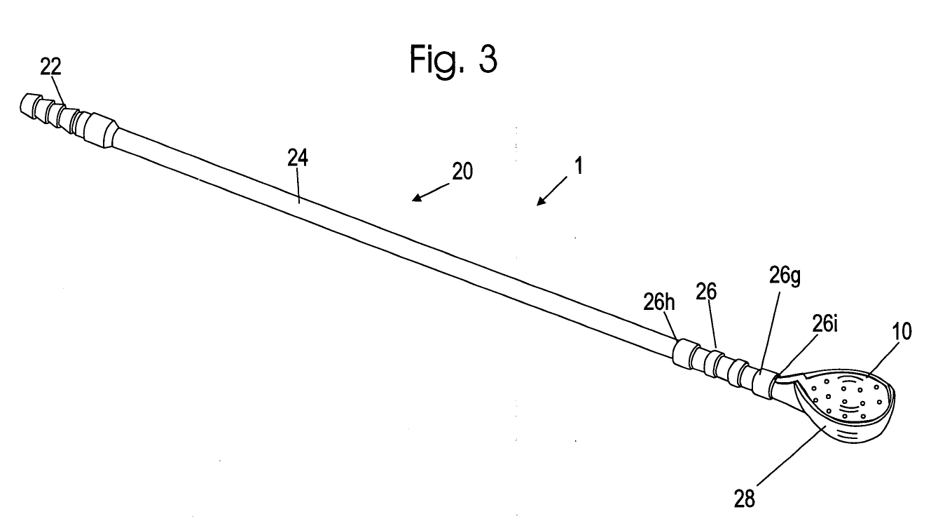

FIG. 2 shows a perspective view of the apparatus 1 of FIG. 1 in an assembled state. In order to assemble the perforated top 10 with the spoon 20, the sleeve 26 is placed or slid to a position, such as the position shown in FIG. 1, such that the sleeve 26 does not block the slot 28a. Next the protrusion 14d of the bowl portion of the perforated top 10, is inserted into the indentation 28g, shown in FIG. 5, in the surface 28e of the bowl device 28. The perforated top 10 is then lowered over the spoon 20 as shown by FIGS. 1 and 2, so that the substantially flat portion 12b of the perforated top 10, lies inside of the slot 28a, so that the bowl portion 14 of the perforated top 10 lies over the bowl portion 28c of the spoon 20, and so that the protrusion 14d lies inside of the indentation 28g in the surface 28e of the bowl device 28. The perforated top 10 can be locked onto the spoon 20 by sliding the sleeve 26 over the substantially flat portion 12b of the stem 12, to the position shown in FIG. 3. In the position of FIG. 3, the stem 12 is held down by the sleeve 26 while at the opposite end the protrusion 14d is held in the indentation 28g of the bowl device 28 so that the perforated top 10 cannot be taken off of the spoon 20 without sliding the sleeve 26 off of the stem 12.

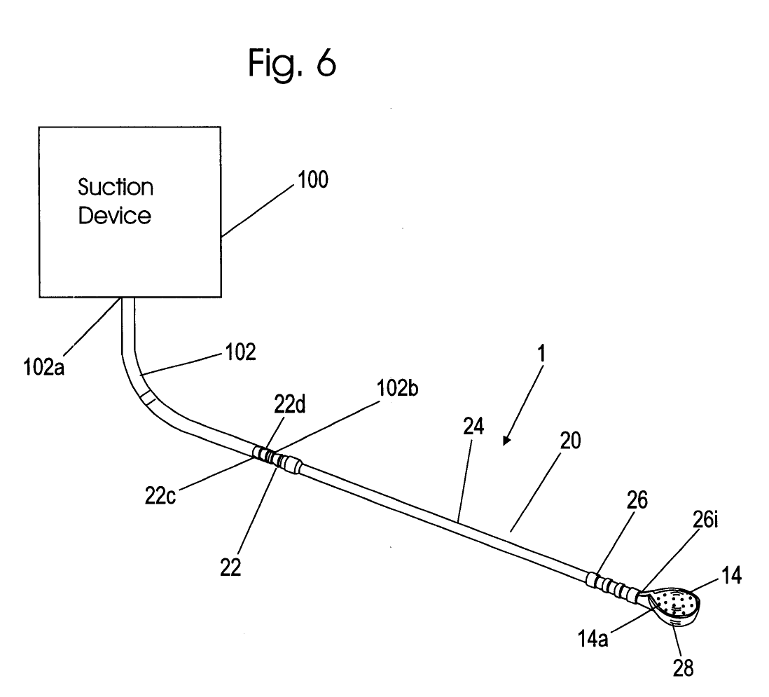

In the state of FIG. 2, an outer portion of the bowl portion 14 of the perforated top 10 rests on the inner ridge 28d so that the bowl portion 14 lies over the bowl portion 28c, but does not contact the bowl portion 28c. Thus, in the state of FIG. 2, there is a space or cavity underneath the bowl portion 14 and over the bowl portion 28c. A liquid, such as water, can fall through the plurality of perforations or holes, such as hole 14a, in the perforated top 10, down into the bowl portion 28c. The liquid can then be suctioned out, through the opening 28b, into the tube 20, into the hollow inside of the connection device 22, and into a suction tube such as tube 102 shown in FIG. 6. The liquid can then be suctioned into a suction device such as suction device 100 of FIG. 6.

FIG. 3 shows a perspective view of the apparatus 1 of FIG. 1 in an assembled state and with the sleeve 26 moved into a position in which it holds the perforated top 10 onto the spoon 20. In the state of FIG. 3, the portion 26g of the sleeve 26 has been moved over the substantially flat portion 12b. This prevents the perforated top 10 from being removed from the spoon portion 20.

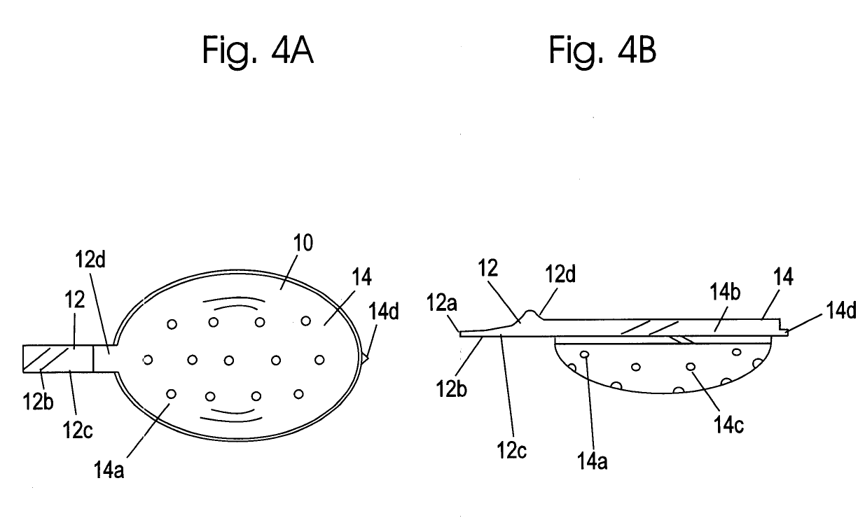

FIG. 4A shows a top view of the perforated top 10. FIG. 4B shows a side view of the perforated top 10.

FIG. 6 shows a perspective view of the apparatus 1 of FIG. 3, attached to the tube 102, which is attached to the suction device 100. The tube 102 includes an end 102a attached to the suction device 100 and an end 102b attached to the connection device 22 of the apparatus 1. The barbs or portions 22c and 22d hold the tube 102 near end 102b to the connection device 22. The tube 102 would typically have an inner diameter which is slightly less than the greatest outer diameter of the barbs or portions 22c and 22d, so that the tube 102 will be expanded by being pushed onto the barbs 22c and 22d and thus will be held onto the barbs or portions 22c and 22d.

The apparatus 1 permits fluids to be suctioned as solid material is removed from an organ or hollow structure during a surgical procedure. This reduces surgical time. In addition the bowl shaped concave surface 14c of the bowl portion 14, better conforms to the shape of human organ objects, which allows these objects to be more readily be removed. The abdominal cavity omentum can be suctioned, while fat can be pushed aside with the non suctioning back surface of the bowl portion 28c, allowing fluids to be removed easier and without clogging of the suction area (perforated top 10 and spoon 20) with abdominal fat or omentum. This allows the surgeon continuous suction without having to stop to clean these things from the apparatus thus reducing surgical time.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. An apparatus comprising:
a perforated top including a bowl portion having a plurality of perforations;
a spoon including a tube portion and a bowl device connected to the tube portion;
wherein the bowl portion of the perforated top is adapted to be placed over the bowl device of the spoon and connected to the bowl device in a manner, which allows liquid to pass through the plurality of perforations, into a portion of the bowl device, and thereafter into the tube portion of the spoon.

2. The apparatus of claim 1 wherein
the perforated top includes a stem connected to the bowl portion;
the spoon includes a slot; and
wherein the stem is adapted to fit within the slot when the bowl portion of the perforated top is placed over the bowl device of the spoon and when the bowl portion of the perforated top is connected to the bowl device.

3. The apparatus of claim 2 wherein
the spoon includes a sleeve, which is adapted to slide over a portion of the stem of the perforated top in order to hold the perforated top onto the spoon.

4. The apparatus of claim 1 wherein
the spoon includes a connection device connected to the tube portion; and
wherein the connection device is adapted to connect the spoon to a suction tube.

5. The apparatus of claim 4 further comprising
the suction tube.

6. The apparatus of claim 5 further comprising
a suction device connected to the suction tube.

7. The apparatus of claim 1 wherein
a protrusion is connected to the bowl portion of the perforated top;
and wherein the bowl device of the spoon includes an indentation into which the protrusion can be inserted in order to attach the perforated top to the spoon.

8. The apparatus of claim 3 wherein
a protrusion is connected to the bowl portion of the perforated top;
and wherein the bowl device of the spoon includes an indentation into which the protrusion can be inserted in order to attach the perforated top to the spoon.

9. A method comprising
providing a perforated top including a bowl portion having a plurality of perforations;
providing a spoon including a tube portion and a bowl device connected to the tube portion; and
wherein the bowl portion of the perforated top is adapted to be placed over the bowl device of the spoon and connected to the bowl device in a manner, which allows liquid to pass through the plurality of perforations, into a portion of the bowl device, and thereafter into the tube portion of the spoon.

10. The method of claim 9 wherein
the perforated top includes a stem connected to the bowl portion;
the spoon includes a slot; and
wherein the stem is adapted to fit within the slot when the bowl portion of the perforated top is placed over the bowl device of the spoon and when the bowl portion of the perforated top is connected to the bowl device.

11. The method of claim 10 wherein
the spoon includes a sleeve, which is adapted to slide over a portion of the stem of the perforated top in order to hold the perforated top onto the spoon.

12. The method of claim 9 wherein
the spoon includes a connection device connected to the tube portion; and
wherein the connection device is adapted to connect the spoon to a suction tube.

13. The method of claim 12 further comprising
connecting the suction tube to the connection device of the spoon.

14. The method of claim 13 further comprising
connecting the suction tube to a suction device.
